(19) United States
(12) Patent Application Publication
(10) Pub. No.: US 2016/0058850 A1
(43) Pub. Date: Mar. 3, 2016

(54) COMPOSITIONS AND METHODS FOR TREATING INFLAMMATORY DISEASES OF INFECTIOUS AND NON-INFECTIOUS ORIGIN

(71) Applicant: BIOGENIUS LLC, Moscow (RU)

(72) Inventors: Anton IGONIN, Gleichen (DE); Paul HERSCU, Amherst, MA (US); Natalya LAZAREVA, Moscow (RU); Valentin URAROV, Moscow (RU)

(21) Appl. No.: 14/436,062

(22) PCT Filed: Nov. 13, 2013
(86) PCT No.: PCT/IB2013/002908

§ 371 Date: Apr. 15, 2015

Related U.S. Application Data
(60) Provisional application No. 61/725,769, filed on Nov. 13, 2012.

(51) Int. Cl. A61K 38/57; C07K 14/81; G01N 33/68; A61K 45/06

(52) U.S. Cl. CPC A61K 38/57; A61K 45/06; C07K 14/8121; G01N 33/6872; G01N 2333/8121; G01N 2800/52

(57) ABSTRACT

The present invention is based, in part, on our analysis of C1-INH levels in various patient populations. Accordingly, in a first aspect, the invention features methods for assessing the protective capacity of endogenous C1-INH in a patient who has been diagnosed with ARDS, sepsis or a sepsis-related condition, a burn or a burn-related condition, SJS, CABG-related states and/or other traumatic injuries. The methods can include the steps of: (a) providing a fluid sample from the patient; (b) determining the amount of C1-INH functional activity in the sample; and (c) comparing the amount of C1-INH functional activity to a reference standard. Where the level of C1-INH functional activity is comparable to that within a healthy patient population, the patient's own protective capacity is compromised.

### CROSS-REFERENCE TO RELATED APPLICATIONS

This application is a 371 U.S. National Phase Application of PCT/IB2013/002908, filed on Nov. 13, 2013, which claims the benefit of the filing date of U.S. Application No. 61/725,769, filed Nov. 13, 2012. The content of this earlier-filed applications is hereby and herein incorporated by reference in its entirety.

### FIELD OF THE INVENTION

The present invention relates to compositions and methods for evaluating and treating patients who are suffering from an inflammatory disease or condition including, but not limited to, acute respiratory distress syndrome (ARDS), sepsis and sepsis-related conditions, burns and burn-related conditions, Stevens-Johnson syndrome (SJS), and coronary artery bypass graft (CABG)-related states. More particularly, the invention includes compositions and methods related to the administration of the C1-esterase inhibitor (C1-INH) either alone or in combination with one or more active pharmaceutical ingredients.

### SUMMARY OF THE INVENTION

The present invention is based, in part, on our analysis of C1-INH levels in various patient populations. Accordingly, in a first aspect, the invention features methods for assessing the protective capacity of endogenous C1-INH in a patient who has been diagnosed with ARDS, sepsis or a sepsis-related condition, a burn or a burn-related condition, SJS, CABG-related states and/or other traumatic injuries. The methods can include the steps of: (a) providing a fluid sample from the patient; (b) determining the amount of C1-INH functional activity in the sample; and (c) comparing the amount of C1-INH functional activity to a reference standard. Where the level of C1-INH functional activity is comparable to that within a healthy patient population, the patient's own protective capacity is compromised. As a reference standard, the C1-INH functional activity can be 1.70 U/L, and the patient's own protective capacity would therefore be considered compromised where the patient's C1-INH activity is about less than or equal to 1.70 U/L (e.g., less than or about equal to 1.34 U/L or between about 1.35 to 1.69 U/L). Where compromise is detected, the present methods can further include the step of administering a therapeutically effective amount of C1-INH to the patient (e.g., a patient who was diagnosed with ARDS, sepsis or a sepsis-related condition, a burn or a burn-related condition, SJS, CABG-related states and/or another traumatic injury). The therapeutic amount of C1-INH administered can vary and can be, in various embodiments: about 12,000 IU administered as two 6,000 IU doses within about a 24-48 hour period; or about 3,000 IU, 6,000 IU, 9,000 IU, 12,000 IU, or 16,000 IU in single or divided doses. More particularly preferred amounts of C1-INH are about 1,000, 2,000, or 3,000 IU administered about every 12 hours. Other preferred amounts of C1-INH are 60-250 U/kg as a single dose or about 1.25-3 U/kg/hour administered as an infusion.

In any embodiment, C1-INH can be administered with a second pharmaceutical agent to a patient (e.g., a patient who has been evaluated as described herein). For example, C1-INH can be administered with protein C, activated protein C, antithrombin III, rituximab, eculizumab, IVIG, complement receptor agonists or antagonists, kallikrein inhibitors or bradykinin receptor inhibitors, antibiotics, steroids, or fresh frozen plasma. The combination therapies disclosed may be administered as one or more pharmaceutical compositions and, if separately, may be administered simultaneously or sequentially in any order.

In any embodiment, the diagnostic or prognostic methods described herein can include a step of assessing the ratio of partial pressure arterial oxygen to the fraction of inspired oxygen (PaO2/FiO2 level), and such a step can be carried out prior to treatment or periodically during the course of treatment. Accordingly, the invention features methods of assessing a patient who has been diagnosed with acute respiratory distress syndrome (ARDS) by: (a) assessing the protective capacity of endogenous C1-INH in the patient, wherein the assessment of C1-INH comprises (i) providing a fluid sample from the patient and (ii) determining the amount of C1-esterase inhibitor (C1-INH) functional activity in the sample; (b) assessing the severity of hypoxemia in the patient, wherein the assessment of hypoxemia comprises measuring the ratio of partial pressure arterial oxygen to the fraction of inspired oxygen (PaO2/FiO2 level); and (c) comparing the amount of C1 INH functional activity to a reference standard. A level of C1 INH functional activity comparable to that within a healthy patient population and a PaO2/FiO2 level below 300 mm Hg indicate that the patient's own protective capacity and gas oxygenation are compromised. The amount of C1 INH functional activity can be less than or about 1.70 U/L, less than or about 1.34 U/L, or about 1.34 to 1.70 U/L, and in each instance the PaO2/FiO2 level can be below 300 mm Hg (e.g., below 200 mm Hg or below 100 mg Hg).

In a second aspect, the invention features pharmaceutical compositions wherein the active pharmaceutical ingredient is C1-INH or a purified biologically active fragment of C1-INH (e.g., the serpin domain or the N-terminal domain). These compositions can be used to treat a patient as described herein (e.g., a patient diagnosed as having ARDS) regardless of the level of C1-INH activity in the patient. For example, the patient can have a C1-INH functional activity above or below about 1.7 U/L. A patient as described herein can also be treated with a C1-INH protein having an inactivated serpin domain, and pharmaceutical compositions including such proteins are within the scope of the present invention. Accordingly, in a third aspect, the invention features methods of treating a patient who has been diagnosed as having ARDS, sepsis or a sepsis-related condition, a burn or a burn-related condition, SJS, CABG-related states and/or other traumatic injuries by administering a therapeutically effective amount of C1-INH or a purified biologically active fragment of C1-INH, either alone or in combination with a second agent, as described herein.

In a fourth aspect, the invention features kits to facilitate the present methods. The kit can include instructions for use in methods of assessing the protective capacity of endogenous C1-INH in a patient (including a patient having any one of the conditions described herein) and one more of the following items: (a) a sample (e.g., a fluid or lyophilized sample) comprising a reference standard; (b) C1-INH; (c) a second therapeutic agent; and (d) paraphernalia for assessing C1-INH and/or delivery of C1-INH and/or the second therapeutic agent to the patient (e.g., medical gloves, swabs, sterilizers, needles, syringes, bandages, reagents, and the like).

The invention is not limited to therapeutic compositions and methods that exert their effect through any particular underlying mechanism of action. We note, however, that the lungs may be protected in the event of ARDS, even in mild form, by limiting either local or systemic inflammatory responses (or both), by ameliorating gas oxygenation and hypoxia, and by controlling vascular permeability (e.g., via control of capillary leakage syndromes and ischemic-reperfusion disorders). The Examples below demonstrate the efficacy of C1-INH in sepsis, severe sepsis, and ARDS, and the present invention developed, in part, from these studies. With regard to medical diagnoses, the compositions and methods of the invention can be employed to assess and monitor inflammatory diseases of infectious and non-infectious origin. Based on an assessment of C1-INH levels, one can determine or predict the efficacy of treatment with C1-INH and, thereby, identify the subset of patients most likely to benefit from C1-INH treatment.

### BRIEF DESCRIPTION OF THE DRAWINGS

FIG. 1 is a schematic illustrating the design of the study described in Example 1.

FIG. 2 is a bar graph illustrating the variability of C1-INH activity (U/L) in patients with sepsis represented in quartiles (Q1, Q2, Q3, and Q4) relative to that in healthy volunteers.

### DETAILED DESCRIPTION

Many diseases that do not share a similar etiology, major risk factor profile, or clinical presentation may in fact result from a shared pathological pathway and/or physiological mechanism. Our hypothesis has been that interactions on the molecular level of one or more of the following systems—the complement, contact, coagulation and immune systems—are implicated in the pathophysiology of such distinctive conditions as ARDS, sepsis and sepsis-related conditions, burns and burn-related conditions, SJS and CABG-related complications. (The condition previously referred to as Acute Lung Injury or ALI is now classified as mild ARDS and is also subject to the methods described herein.) Physiological systems are obviously complex and many molecules are likely to be involved in coordinating an interaction among such systems. On the other hand, single endogenous regulators may activate or inhibit the functioning of one or all of the systems referred to above.

C1-INH is a unique protein with a wide range of biological properties. It is currently being studied extensively, and we believe it will have a profound impact on patients suffering from the conditions described herein, regardless of their diversity. C1-INH belongs to a superfamily of serine protease inhibitors, and it acts as an acute-phase protein and endogenous regulator of both the complement and kallikrein-kinin systems by inhibiting complement (C1r, C1s, mannan-binding lectin-associated serine protease-2), contact (factor XII, kallikrein), and coagulation proteases (factor XI; van der Graaf et al., J. Clin. Invest. 71:149-158, 1983; Caliezi et al., Pharmacol. Rev. 52:91-112, 2000; and Kalter et al., J. Infect. Dis. 151:1019-1027, 1985).

Endogenous human C1-INH is a glycoprotein including 478 amino acids, and it belongs to the serpin superfamily of proteins. It is a single chain protein with a molecular weight of 105 kDa. It also has a two-domain structure, which distinguishes it from other serpin proteins. The C-terminal domain provides the serine-protease inhibitory activity and the N-terminal domain is heavily glycosylated. Protease recognition, binding and further inactivation is predominantly the responsibility of the serpin C-terminal domain. C1-INH inhibits a wide range of proteases of the complement system (C1r and C1s, MASP1 and MASP2), contact system (XII, kallikrein), coagulation system (XI, thrombin) and fibrinolytic system (tPA and plasmin). Although the full spectrum of N-terminal domain mediated mechanisms is unknown, several anti-inflammatory properties such as leucocytes adhesion, neutrophil accumulation, and LPS interaction might be attributed to it.

C1-INH modulates the coagulation cascade, impacting leukocyte activation (Wuillemin et al., Blood 85:1517-1526, 1995; and Eriksson and Sjögren, Immunology 86:304-310, 1995), enhancing bactericidal activity, and preventing endotoxin shock via direct stimulation with bacterial lipopolysaccharide, as demonstrated in a sepsis model (Liu et al., Blood 105:2350-2355, 2005). In a human experimental endotoxemia study, high doses of C1-INH exerted an anti-inflammatory effect independent of the classic complement pathway (Dorresteijn et al., Crit. Care Med. 38:2139-2145, 2010).

Infectious agents, regardless of the initial, primary site of infection, trigger local inflammatory reactions that are often contained. In some instances, however, infectious agents activate systemic immune and cellular responses that affect a patient's entire body. When a localized infection progresses in this way, the patient has a potentially fatal condition that manifests as systemic inflammation, often called sepsis or, more colloquially, “blood poisoning.” Microbes or pathogen-associated molecular patterns (PAMPs) present in the blood, urine, lungs, skin, or other tissues trigger an immune response, and sepsis advances to severe sepsis where the immune and inflammatory response is great enough to cause organ dysfunction. Severe sepsis may progress to septic shock. Systemic inflammatory response syndrome (SIRS), related to sepsis, is an inflammatory state that affects the entire body and is often a response of the immune system to an infection. The American College of Chest Physicians and the Society of Critical Care Medicine have defined SIRS as a condition in which two or more of the following symptoms are present: altered body temperature (hypothermia or fever); elevated heart rate; elevated respiratory rate; and altered white blood cell counts (leukopenia, leukocytosis, or bandemia; see Bone et al., Chest 101(6):1655-1655, 1992). Infection can be confirmed by culturing and testing a specimen obtained from the patient. Specific signs of infection include the presence of white blood cells in normally sterile bodily fluid (e.g., urine or cerebrospinal fluid); a perforated viscus (free air in images of the abdomen and/or signs of acute peritonitis); an abnormal chest x-ray (e.g., consistent with pneumonia); or petechiae, purpura, or purpura fulminans. Severe sepsis is defined as sepsis with organ dysfunction, hypoperfusion, or hypotension, and septic shock is defined as sepsis with refractory arterial hypotension or hypoperfusion abnormalities in spite of adequate fluid resuscitation. The transformation of a local infection to a systemic response is profound and may greatly impact the clinical outcome.

Sepsis can develop and progress in healthy people, but the incidence is higher and the prognosis is worse for compromised patients, including those who are chronically ill and/or immuno-compromised. Severe sepsis usually requires treatment in an intensive care setting with intravenous fluids and possibly vasopressors to maintain blood pressure and antibiotics to fight the underlying infection. Patients may also require mechanical ventilation, dialysis, and central venous or arterial catheterization. Sepsis is a leading cause of mortality and death in developing countries as well as in advanced care units in the developed nations. Mortality rates have not dropped despite the availability of new therapeutic modalities. Delay and inappropriate empirical antibiotic treatments are associated with worse outcomes. In the meta-analysis of Friedman, sepsis mortality ranged from 40% to 80% (Friedman et al., Crit. Care Med. 26:2078-2086, 1998). The average mortality rate for patients with severe sepsis in the United States has been reported as 28.6% (Angus et al., Crit. Care Med. 29:1303-1310, 2001). These numbers indicate the limited effectiveness of antibiotics. It may be that, after a certain point in time, antibiotics are not able to impede progression of the condition or syndrome, and a dysregulated immune system causes significant damage.

Several conditions are caused by or related to sepsis, however, many of these conditions can also arise independent of sepsis. The conditions described herein can be assessed and treated according to the present methods regardless of whether or not they are related to sepsis or of completely independent origin. For example, ARDS can have an infectious or non-infectious origin. In the lungs, excessive localized reactions lead to capillary and alveolar damage with an accompanying increase of extravascular fluid as well as massive inflammation and local coagulopathy. These are among the key pathological features of ARDS. ARDS was first described by Ashbaugh in 1967 (Ashbaugh et al., Lancet 2(7511):319-323, 1967). ARDS continues to plague critical care facilities, with the incidence in the United States reported to be about 50,00-190,000 cases per year (Rubenfeld et al., N. Engl. J. Med. 353(16):1685-1693, 2005; Goss et al., Crit. Care Med. 31(6):1607-1611, 2003). Since ARDS was first described, significant research has helped to elucidate its underlying pathophysiology, course, and genetic predisposition, and mechanical ventilation methods have improved treatment outcomes and survival rates over time (Amato et al., N. Engl. J. Med. 338(6):347-354, 1998; Anonymous, N. Engl. J. Med. 342(18):1301-1308, 2000; Zambon and Vincent, Chest 133(5):1120-1127, 2008; Gao and Barnes, Am. J. Physiol. Lung Cell Mol. Physiol. 296(5):L713-725, 2009; Gagger and Olman, Clin. Chim. Acta 372(1-2):24-32, 2006; and Lam and dos Santos, Curr. Opin. Crit. Care 14(1):3-10, 2008). Despite clinical advances, ARDS remains a life-threatening complication of sepsis. Indeed, ARDS was verified in almost 18-38% of patients with sepsis regardless of the primary infectious foci (Fein and Calalang-Colucci, Crit. Care Clin. 16(2):289-317, 2000).

ARDS is associated with a variety of pathological findings. The accumulation of bradykinin and thrombin, along with endothelial alveolar damage, is thought to contribute significantly to vascular permeability and alveolar edema. For instance, thrombin interacts with the PAR-1 receptor to induce a cytosolic shift in Ca²⁺ in pulmonary artery endothelial cells that results in increased permeability (Garcia et al., J. Cell Physiol. 156(3):541-549, 1993; Vogel et al., Physiol. Genomics 4(2):137-145, 1994). Although bradykinin plays a major role in angioedema due to C1-INH deficiency (Shoemaker et al., Clin. Exp. Immunol. 95(1):22-28, 1994), the role of the contact system in pulmonary edema in ARDS remains unclear. Important regulators of bradykinin include components of the renin-angiotensin system and may be involved in ARDS pathogenesis. Recently, it was shown that the angiotensin converting enzyme (ACE) 2 could act as a lung protector, mediating vascular leakage in ARDS during SARS (Li et al., Nature 426(6965):450-454, 2003; Imai et al., Nature 436(7047):112-116, 2005). Polymorphisms in ACE may affect mortality rates (see Rigat et al., J. Clin. Invest. 86(4):1343-1346, 1990; and Adamzik et al., Eur. Respir. J. 29(3):482-488, 2007). Down-regulation of such endogenous signaling pathways may protect tissues and organs from fatal injury. Presently, however, there is no modulator capable of limiting host-mediated damage and improving negative survival trends in both sepsis and ARDS.

An uncontrolled inflammatory response can disrupt the function of organ systems in the body and in some cases lead to medical intervention to maintain homeostasis in an ill patient. A change in the function of two or more organ systems requiring medical attention to maintain stable functioning is referred to as multiple organ failure. Conditions that may lead to an uncontrolled inflammatory response include but are not limited to the following: sepsis, sepsis-related conditions, burns and burn-related conditions, SJS and CABG-related states. These conditions may lead to the dysfunction of one or more of the following organ systems: the respiratory system (respiratory dysfunction), the central nervous system (encephalopathy), the hepatic system (disruption of protein function), the renal system (oliguria, anuria, electrolyte imbalance, volume overload) and the cardiovascular system (hypotension, lactic acidosis, oliguria, prolonged capillary refill, metabolic acidosis and other forms of cardiovascular dysfunction). Burns that affect deeper tissues such as muscles, bones and blood vessels may lead to the dysfunction of multiple organ systems. Further, even with managed care, burn injuries may be associated with complications including sepsis, septic shock, infection, electrolyte imbalance and respiratory distress. SJS cases, on the other hand, are in large part idiopathic. They may result from dysfunction of the immune system and may be linked to an infection. The use of certain medications may also trigger an immune reaction that leads to organ dysfunction. Patients undergoing CABG surgery have an increased risk for developing infections and, like the other patients described herein, can be subjected to the present diagnostic and therapeutic methods.

Purified human C1-INH is useful as a replacement therapy in hereditary angioedema (HAE). It has been used for decades in prophylactic and acute treatment of HAE at recommended doses of 20 U/kg (Frank and Jiang, J. Allergy Clin. Immunol. 121:272-280, 2008; Gadek et al., N. Engl. J. Med. 302:542-546, 1980; Bergameschini et al., J. Allergy Clin. Immunol. 83:677-682, 1989; Horstick et al., Circulation 104:3125-3131, 2001). As highlighted in 2001, a potential problem occurred when C1-INH was administered in >100 U/kg, quelling interest in using high-does C1-INH as a treatment for other illnesses. Specifically, procoagulant side effects were reported in coronary occlusion models in non-heparinized animals (Horstick et al., Circulation 104:3125-3131, 2001). Other authors, however, have since reported effective complement activation, reduction of myocardial injury, renal protection effect, decreased demands in vasopressors, and an absence of significant adverse effects in those critical care patients receiving high-dose C1-INH infusion (Caliezi et al., Crit. Care Med. 30:1722-1728, 2002; and de Zwaan et al., European Heart J. 23:1670-1677, 2002).

Recent studies with other therapeutic approaches have demonstrated increased survival rates in sepsis patients once relevant criteria for a latent sub-group analysis was introduced (Bernard et al., N. Engl. J. Med. 344:699-709, 2001; and Laterre et al., Crit. Care Med. 35:1457-1463, 2007). We, therefore, hypothesized that the heterogeneity of sepsis patients might conceal potentially advantageous effects of administration of high concentrations of C1-INH. In Example 1 below, we analyzed population subsets according to sepsis severity parameters and assessed the benefits of high-dose C1-INH infusion.

Diagnostic and Prognostic Methods:

The methods of the present invention include diagnostic methods in which one assesses the degree of risk that an inflammatory process will progress to a more severe, and perhaps even a life-threatening, condition. In these methods, assays are carried out to measure functional and/or nonfunctional C1-INH protein levels and/or expression or activity levels in a patient. For example, a patient diagnosed with ARDS, sepsis or a sepsis-related condition, a burn or a burn-related condition, SJS and/or a CABG-related disorder. In some embodiments, the diagnostic methods described herein are carried out on patients who do not have a sign or symptom of one of these conditions. The patient can be one who is apparently in good health. The results of this test can serve as a personal reference standard for the patient if needed at a later point in time. Specimens containing serum or EDTA plasma samples are collected from the patients under aseptic conditions and prepared using techniques for clinical laboratory testing; the kits described herein can facilitate this sample collection as well as preparation and testing. The functional activity of C1-INH can be determined using a variety of assays, including any commercially available chromogenic assay (e.g., Berichrom C1-inhibitor, Siemens, Germany) and then compared to a manufacturer's standard. Steady, non-changing levels of C1-INH activity and/or “normal” protein activity levels in the patient (a patient having a condition as described herein, for example) in the course of their disease will indicate that the protective capacity of the protein is compromised, placing the patient at risk for developing an increasingly severe systemic inflammatory response. Generally, C1-INH levels below the reference standard (e.g., during acute inflammation) indicate that C1-INH functional activity is compromised, leading to a more severe state, while C1-INH levels above the reference standard indicate a normal, systemic inflammatory response. Treatment with C1-INH either alone or in combination with one or more active pharmaceutical agents is recommended in patients with C1-INH levels that are about equal to or below the reference standard, and such treatments are within the scope of the present invention. Treatment is also recommended in patients with C1-INH levels of about 1.7 U/L or less or in patients with steady, non-changing levels of C1-INH along with increased levels of CRP (45-415 mg/L), indicating a sustained systemic inflammatory response. Thus, the methods of the invention can also include a step of analyzing CRP levels. As noted above, any of the present diagnostic or prognostic methods can also include a step of assessing the severity of hypoxemia in the patient, and that assessment can include measuring the ratio of partial pressure arterial oxygen to the fraction of inspired oxygen (PaO₂/FiO₂ level). Levels below 300, 200, or 100 mm Hg indicate increasingly severe hypoxemia.

The patient can be of any age. Accordingly, the patient can be an infant, child, adolescent, adult, or elderly patient. The patient can also be an individual who was either previously in good health (i.e., in good health prior to experiencing a condition as described herein) or chronically ill and/or immuno-compromised prior to experiencing a condition as described herein.

Conditions Amenable to Evaluation and Treatment:

The conditions that can be evaluated and treated as described herein include inflammatory diseases of infectious and non-infectious origin. Where the condition either evolves from sepsis or predisposes a patient to sepsis, we may refer to it as a “sepsis-related condition.” For example, sepsis, severe sepsis, septic shock and SIRS are all sepsis-related conditions. As noted above, conditions such as ARDS can have an infectious origin, and when they do arise from an infection that progresses to sepsis, these conditions are also “sepsis-related conditions.” Also as noted above, burns, particularly severe burns, may lead to the dysfunction of multiple organ systems and are associated with complications including sepsis, septic shock, infection, electrolyte imbalance and respiratory distress. These downstream consequences are “burn-related” conditions. While we tend to use the term “condition,” we may also use terms such as “disorder” or “disease” without appreciable difference.

Conditions amenable to evaluation and, if merited, treatment, with C1-INH include, without limitation, septic shock, ARDS, and Stevens-Johnson Syndrome. The methods can also be applied where a patient has a burn or other traumatic injury (whether caused accidentally or in the course of a surgical procedure, such as an organ, tissue, or cell transplant). Patients are at a greater risk of having an inflammatory process which may lead to a life-threatening condition, following such insults.

Pharmaceutical Formulations, Doses, and Administration:

Pharmaceutical compositions for use in accordance with the present invention may be formulated using one or more physiologically acceptable carriers or excipients. Any suitable concentration of C1-INH may be used, and that active pharmaceutical ingredient will be administered in an amount effective to achieve its intended purpose. We may refer to such an amount as a “therapeutically effective amount.” Determination of a therapeutically effective amount of C1-INH or a second active ingredient is within the capability of one of ordinary skill in the art.

The specific, therapeutically effective dose level for any particular patient will depend upon a variety of factors including the amount and activity of the C1-INH protein; the activity of any other specific compounds employed; the specific composition employed; the age, body weight, general health, sex and diet of the patient; the age of the patient; the time of administration, route of administration, and rate of excretion of C1-INH; the duration of the treatment; drugs used in combination or coincidentally with the C1-INH; and like factors well known in the medical arts.

The therapeutically effective dose of C1-INH can be administered using any medically acceptable mode of administration. Moreover, in the event of a combination therapy, a C1-INH inhibitor can be administered with a second agent in a single dosage form or otherwise administered in combination (e.g., by sequential administration through the same or a different route of administration). Although one would contemplate any of the modes of administration known in the art, preferably the pharmacologic agent is administered according to the recommended mode of administration, for example, the mode of administration listed on the package insert of a commercially available agent. In general, the dose may comprise 1,000-20,000 IU doses in single or divided doses within a 24-48 hour period and 1.25-250 U/kg in a single dose or per hour.

Therapeutic agents, for example inhibitors of complement, etc. can be incorporated into a variety of formulations for therapeutic administration by combination with appropriate pharmaceutically acceptable carriers or diluents, and may be formulated into preparations in solid, semi-solid, liquid or gaseous forms, such as tablets, capsules, powders, granules, ointments, solutions, suppositories, injections, inhalants, gels, microspheres, and aerosols. As such, administration of the compounds can be achieved in various ways, including oral, buccal, rectal, parenteral, intraperitoneal, intradermal, transdermal, intrathecal, nasal, intracheal, etc., administration. The active agent may be systemic after administration or may be localized by the use of regional administration, intramural administration, or use of an implant that acts to retain the active dose at the site of implantation.

Pharmaceutical compositions described herein may be administered directly, they may also be formulated to include at least one pharmaceutically-acceptable, nontoxic carriers of diluents, adjuvants, or non-toxic, nontherapeutic, fillers, buffers, preservatives, lubricants, solubilizers, surfactants, wetting agents, masking agents, coloring agents, flavoring agents, and sweetening agents. Also, as described herein, such formulation may also include other active agents, for example, other therapeutic or prophylactic agents, nonimmunogenic stabilizers, excipients and the like. The compositions can also include additional substances to approximate physiological conditions, such as pH adjusting and buffering agents, toxicity adjusting agents, wetting agents and detergents.

Methods of making a pharmaceutical composition include admixing at least one active compound, as defined above, together with one or more other pharmaceutically acceptable ingredients, such as carriers, diluents, excipients, and the like. When formulated as discrete units, such as tablets or capsule, each unit contains a predetermined amount of the active compound.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

The compositions can be administered to the subject in a series of more than one administration. For therapeutic compositions, regular periodic administration will sometimes be required, or may be desirable. Therapeutic regimens will vary with the agent, e.g. some agents may be taken for extended periods of time on a daily or semi-daily basis, while more selective agents may be administered for more defined time courses, e.g. one, two three or more days, one or more weeks, one or more months, etc., taken daily, semi-daily, semiweekly, weekly, etc.

Suitable formulations will depend on the method of administration. The pharmaceutical composition is preferably administered by parenteral administration, such as for example by intravenous, intra-arterial, subcutaneous, intraperitoneal or intramuscular injection or infusion; or by intrathecal or intracranial administration. In a preferred embodiment it is administered by intravenous infusion. Suitable formulations for parenteral administration are known in the art and are typically liquid formulations. These liquid formulations may for example be administered by an infusion pump.

The effective dose (i.e., the effective concentration and frequency of administration), will depend on the specific pharmaceutical composition which is used, the severity of the condition and the general state of the patient's health. A suitable starting point is the dose which is used for the equivalent pharmaceutical composition which is based on plasma-derived C1-INH. The pharmaceutical compositions can be administered for prophylactic and/or therapeutic treatments.

Combination Therapies:

The present methods can be carried out in combination with other treatments, and the C1-INH-containing compositions described herein can be administered together with other pharmaceutical agents. For ease of administration, the C1-INH and a second pharmaceutical agent can be combined in the same dosage form. The classes of pharmaceutically active agents that may be useful in the present invention include but are not limited to are glycoproteins involved in the coagulation system, monoclonal (chimeric and/or recombinant) antibodies, immunoglobulin antibodies, kallikrein inhibitors, bradykinin receptor inhibitors, antibiotics, steroids, immunomodulators, agonists or antagonists of complement receptors, activators and inhibitors of complement and fresh frozen plasma.

Preferred pharmaceutical agents include but are not limited to: glycoproteins such as antithrombin III (which may be administered as an isolated, natural protein (Antithrombin III Immuno) or as expressed by recombinant methods), protein C and activated protein C (drotrecogin-α); antibodies, including chimeric monoclonal antibodies such as rituximab (Rituxan™, MabThera), and recombinant monoclonal antibodies such as eculizumab (Soliris™) and immunoglobulin antibody G such as intravenous immunoglobulin (IVIG); antibiotics including glycopeptides such as vancomycin (Vancocin™), teicoplanin (Targocid™), telavancin (Vibativ™), bleomycin (Blenoxane™), ramoplanin and decaplanin; cephalosporins generation III such as cefcapene, cefdaloxime, cefdinir (Zinir™, Omnicef™, Kefnir™), cefditoren, cefetamet, cefixime (Zifi™, Suprax™), cefmenoxime, cefodizime, cefotaxime (Claforan™), cefovecin (Convenia™), cefpimizole, cefpodoxime (Vantin™, PECEF), cefteram, ceftibuten (Cedax™), ceftiofur, ceftiolene, ceftizoxime (Cefizox™), ceftriaxone (Rocephin™) cefoperazone (Cefobid™), ceftazidime (Fortum™, Fortaz™), and including the following cephems are often grouped with third-generation cephalosporins, latamoxef (moxalactam); cephalosporins generation IV such as cefclidine, cefepime (Maxipime™), cefluprenam, cefoselis, cefozopran, cefpirome (Cefrom™), cefquinome, and including the following cephems that are often grouped with fourth-generation cephalosporins, flomoxef; and quinolones; kallikrein inhibitors such as aprotinin (Trasylol™) and ecallantide (Kalbitor™, DX-88); bradykinin receptor inhibitors such as icatibant (Firazyr™); agonists or antagonists of complement receptors (CR) including CR1-4 and any subtypes of CR1-4; steroids including corticosteriods such as glucocorticoids (hydrocortisone, cortisone, prednisone, prednisolone, methylprednisolone, dexamethasone, betamethasone, triamcinolone, beclometasone, fludrocortisone acetate, deoxycorticosterone acetate and aldosterone); immunosuppressants including calcineurin inhibitors such as cyclosporine (Ciclosporin™) and tacrolimus (also referred to as FK-506 or fujimycin; Prograf™, Advagraf™, Protopic™); antiproliferative drugs including azathioprine, cyclophosphamide (Endoxan™, Cytoxan™, Neosar™, Procytox™, Revimmune™), methotrexate (Trexall™), chlorambucil (Leukeran™), mycophenolate mofetil (CellCept™, Myfortic™), glucocortocoids (see above) and antibodies (see above) including muromonab CD3, antithymocyte globin, Rho (D) immuneglobin, and efalizumab (Raptiva™); immunostimulants including levamisole (Ergamisol™), thalidomide (Thalomid™), and recombinant cytokines such as interferons and interleukin 2 (aldeslukin); tolerogens; and fresh frozen plasma.

Any of the treatment methods described herein can be expressed in terms of “use”. For example, the invention encompasses the use of C1-INH in the treatment of a condition as described herein (e.g., ARDS) and the use of C-INH in the preparation of a medicament for the treatment of a condition as described herein (e.g., ARDS).

Kits:

The present invention includes a kit for use by a healthcare provider. The kit provides a mechanism for determining the protective capacity of endogenous C1-INH functional activity for the assessment of the degree of severity of systemic inflammation in a patient with an inflammatory conditions such as those described herein (e.g., ARDS, burns, sepsis, SJS and/or CABG related disorders). The kit can be comprised of a commercially available chromogenic assay to first determine C1-INH activity levels from a serum sample, a mechanism for comparing the C1-INH activity levels to a reference standard to determine the degree of severity of systemic inflammation and selecting the appropriate dosing regimen, and, optionally, a pharmaceutical composition comprised of C1-INH at a therapeutically effective dose for administration or means for ordering such a pharmaceutical composition from a pharmacy or other dispensary. The kit of the invention can be comprised of various combinations of reagents, drugs, sample procurement devices, and drug delivery devices. The other, non-C1-INH drug may be given using any medically acceptable mode of administration. Generally, instructions for use are included in the kits.

### EXAMPLES

### Example 1

### C1-Esterase Inhibitor Infusion Increases Survival Rates for Patients with Sepsis

The study described below was designed to assess the influence of high-dose C1-INH on systemic inflammatory responses and survival in patients with sepsis. Patients were randomized to receive either 12,000 U of C1-INH by infusion, together with conventional treatment, or conventional treatment alone.

Study Design:

We carried out an open-label, randomized controlled study in nine independent intensive care units in community and teaching hospitals of Moscow and Saint Petersburg, Russia. We used a block randomization with the treatment arm vs. the control arm having an allocation of 2:1 ratio (treatment:control). The Federal Ministry of Health of the Russian Federation as well as national and local ethics committees approved the two-stage study protocol. Guidelines of Good Clinical Practice were adhered to and fulfilled the requirements of the Declaration of Helsinki. All conscious and able patients included in the study gave written informed consent. Where that was not possible, we obtained consent from a relative or, if relatives could not be found, other legal representatives. Adverse events and severe adverse events were registered during the entire study period of 28 days.

Sepsis, severe sepsis, and septic shock were characterized according to the American College of Chest Physicians/Society of Critical Care Medicine definition (1992), needing three or more systemic inflammatory response syndrome criteria to be fulfilled (American College of Chest Physicians/Society of Critical Care Medicine Consensus Conference: Definitions for sepsis and organ failure and guidelines for the use of innovative therapies of sepsis, Crit. Care Med. 20:864-874, 1992). Procalcitonin above 2 ng/ml was used as a biomarker of infection in cases of acute pancreatitis or bacteremia where there was no definitive primary focus of infection. Eligible patients were included only if they could begin treatment within 48 hours of sepsis onset.

Patients were ineligible if they met any of the following exclusion criteria: an age less than 18 years; HAE; more than three organs/systems dysfunction (i.e., patients were excluded when they were affected by the primary site of infection and two other organs, with the failing systems needing to be new and sepsis related, not explained by the original underlying disease progression); the presence of a terminal stage chronic disease (i.e., congestive heart failure class IV, chronic liver insufficiency Child-Pugh class C, chronic renal failure with hemodialysis); acute myocardial infarction; cardiogenic shock; stroke; life-threatening bleeding during the previous 48 hours; verified or suspected tuberculosis; open head injury during the last month; high probability of any major surgical intervention during the subsequent 28 days; a Glasgow Coma Scale score less than 9; pregnancy; allergy to human plasma proteins; or participation in another clinical study. Organ dysfunction criteria were identified as in Baue et al. (Baue et al. (Eds), Multiple Organ Failure: Pathophysiology, Prevention and Therapy, New York, Springer, 2000).

Clinical and Laboratory Evaluation:

Vital signs were documented and routine hematologic and biochemical assays were carried out on days 1, 2, 3, 5, 7, 10 and 28 after randomization. We assessed illness severity using the Simplified Acute Physiology Score II (SAPS II). Additional diagnostic procedures were carried out at the discretion of the attending physicians. Twenty-eight day mortality was assessed.

Blood samples were drawn on the days indicated above and were studied by immuno-turbidimetry (Architect ci8200, Abbott) to determine serum concentrations of complement components C3 and C4. C1-INH activity during sepsis was measured using a commercial chromogenic assay (Berichrom C1-inhibitor, Dade-Behring, Germany). To assess the correct normal C1-INH plasma range, specimens from ten healthy volunteers were used, along with the manufacturer's standard information (0.7-1.3 U/L). The samples from all sites were analyzed at an independent laboratory.

Treatment:

All patients randomized into the treatment arm received an initial 6000 U infusion of C1-INH (Bicizar, BioGenius, Moscow, Russia). The intravenous infusion was started immediately after randomization once the lyophilized content of each vial was dissolved in 10 mL of 0.9% sodium chloride solution. C1-INH was further diluted in 0.9% sodium chloride solution to reach 10 U/mL C1-INH, the upper concentration limit for the infused solution. Treated patients received a total of 12,000 U of C1-INH, administered over the initial 48 hours. The daily dose of C1-INH was chosen based on the de Zwaan et al. study (European Heart J. 23:1670-1677, 2002).

All patients in the treatment arm received C1-INH alongside standard care. All treatment decisions regarding surgery, administration of antibiotics, fluid infusions, vasopressors, anti-coagulants, and respiratory support were at the discretion of the attending physicians and were not ruled by protocol. Total fluid therapy was adjusted to accommodate for the extra C1-INH solution volume added to the total daily calculations. None of the patients in the trial received extra fluid volume.

Statistical Analysis:

Statistical comparisons were calculated using the Mann-Whitney U test. Associations between variables were assessed by the Spearman's rank correlation coefficient. Qualitative results were analyzed with Fisher's exact test. Survival rates were calculated using the Kaplan-Meier method, and the data presented was presented as median and range. Odds ratio all-cause mortality in subgroups of patients was estimated using Cochran-Mantel-Haenszel statistics, adjusted for the initial imbalance on admission. Receiver operating curve analysis was applied to estimate individual tests' ability to discriminate between the two groups of patients who differed in their response to the therapy on day 28 (McNeil et al., N. Engl. J. Med. 293:211-215, 1975). All statistical tests were carried out using Graph-Pad Prism (GraphPad Software) and SPSS version 16 (SPSS).

Results:

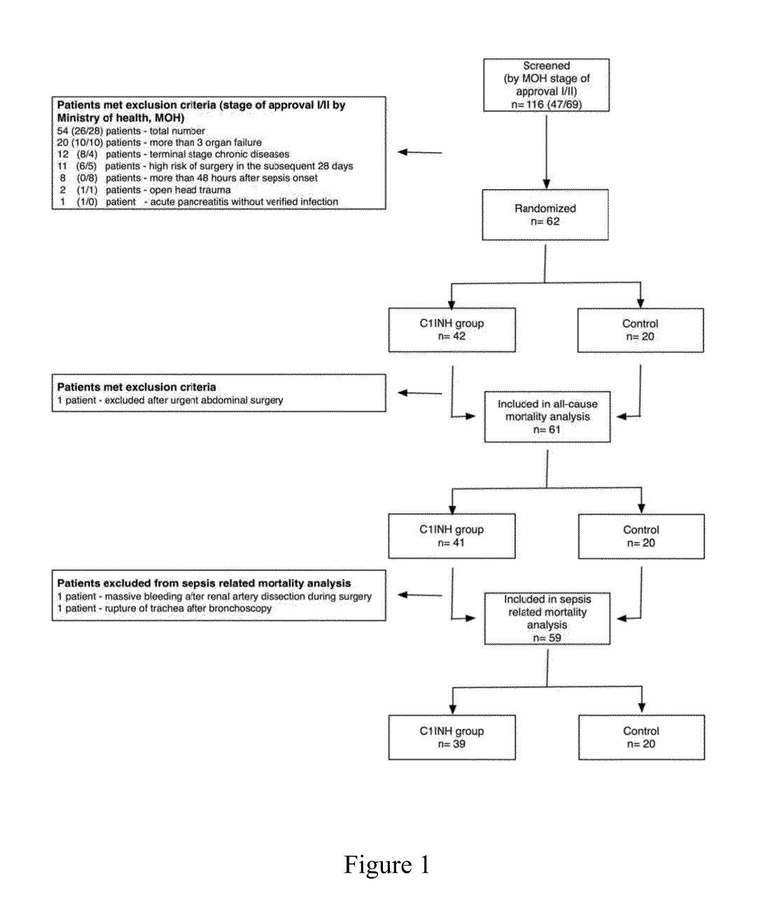

A total of 116 sepsis patients were screened in two stages, with 54 meeting exclusion criteria (FIG. 1). Of the 62 sepsis patients included in the study, 42 were randomized into the treatment group to receive C1-INH infusion, while the remaining 20 were enrolled as control. Soon after inclusion, one treatment group patient unexpectedly underwent urgent abdominal surgery due to an exacerbation of his underlying disease, which satisfied one exclusion criterion and no data from this patient were included in the final analysis. No serious drug-related adverse events were identified in the treatment group.

The baseline demographic and laboratory characteristics were similar between the C1-INH and control groups. There was a prevalence of severe sepsis (64%) among all patients enrolled in the study. No statistical differences were found in SAPS II score, number of mechanically ventilated patients, nor subjects on vasopressors at the study commencement. However, the number of postoperative cases was significantly higher in the control arm (p=0.04). The groups were well balanced with respect to underlying diseases. At study entry, the lungs (35 of 61) and abdomen (14 of 61) were the most common primary sites of infection in both groups.

Microbiological analysis was positive in 56% of the patients (34 of 61). In the treatment group, nine cases of Gram-negative bacteria (Pseudomonas aeruginosa and Klebsiella pneumoniae) were found in sputum/lavage fluid culture and eight isolates were obtained from bloodstreams. In the control group, five patients with Gram-negative bacteria in sputum/lavage fluid culture and one patient with bloodstream Gram-negative bacteria were noted. Concomitant heparin was used in 63% (26 of 41) of the C1-INH group patients and in 50% (ten of 20) of the control (p>0.05). No significant differences between the two arms were found when looking at variables such as ventilator management and hemodynamic support. Steroids were administered to 17% (seven of 41) of the patients in the treatment group and 15% (three of 20) of the control group (p=1.00). Carbapenems as monotherapy or in combination were the most often empirical antibiotic prescription in both groups, with 21 of 41 (51%) for the C1-INH group and 13 of 20 (65%) in the control group (p=0.4).

Two deaths occurred in the C1-INH group, on days 5 and 8 of the study period, and data from these patients were excluded from analysis of sepsis mortality but included in the all-cause mortality rate. In the first case, intrasurgical dissection of the renal artery was followed by massive bleeding. In the second case, severe rupture of the trachea was the cause of death in a mechanically ventilated patient due to diagnostic bronchoscopy.

Baseline C1-INH Activity:

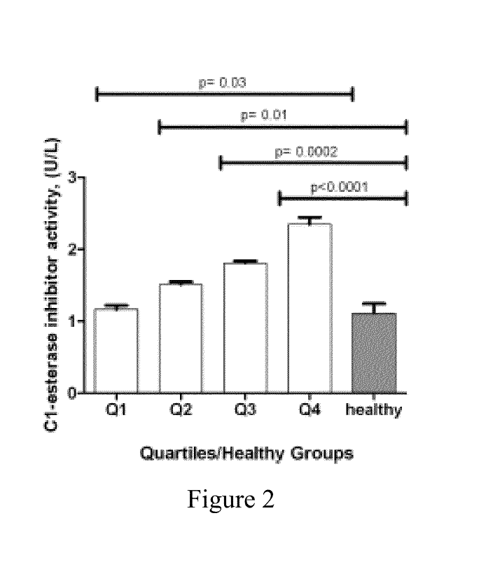

Quartile Analysis: The variability of C1-INH activity in sepsis patients' plasma is shown in FIG. 2. All patients were stratified in quartiles according to the C1-INH activity on day 1. The four quartiles ranged from: Q1, 0.81 to 1.34 U/L (n=17); Q2, 1.35 to 1.69 U/L (n=18); Q3, 1.7 to 1.98 U/L (n=12); and Q4, >1.98 U/L (n=14) of C1-INH activity of healthy volunteers (0.55-1.87 U/L) and sepsis patient from the highest quartiles. The level of C1-INH activity reached 1.84 U/L (1.7-1.98 U/L) in Q3 (p=0.0002) and 2.25 U/L (1.99-2.96 U/L) in Q4 (p<0.0001). These data show that C1-INH activity of the sepsis patients from the lowest quartile (Q1) displayed no statistical difference from the healthy volunteers (p>0.05).

The concentration of C-reactive protein (CRP, an acute-phase protein) was significantly increased in the first quartile of patients; the same patients who had normal C1-INH levels at admission. These patients also tended to have decreased C4 levels compared to patients from the highest quartile on day 1. The percentage of patients with severe sepsis stratified to the lowest quartiles (Q1+Q2) was 77% (27 of 35 patients) whereas 23% (eight of 35 patients) met the criteria of non-severe sepsis (p=0.001).

Inflammatory Markers:

No difference was observed in C1-INH activity between the two arms after randomization or during the initial phase of treatment. Inter-group differences, however, appeared by day 2 (p<0.0001). The significantly elevated C1-INH activity in plasma of C1-INH-treated patients lasted through day 10 (p=0.004). Depleted C3 complement subunit levels were elevated in the treatment group, and restored levels were detected on days 2-3 as a result of increased C1-INH activity (p=0.02). C3 was positively correlated with C4 (r=0.49; p<0.0001). SAPS II score and C4 were inversely correlated with high significance (r=−0.37; v<0.0001) during the first three days of the study.

We observed a significant drop in CRP levels in the C1-INH group on day 3 (p=0.02). This difference in CRP concentration between the patients receiving C1-INH infusions and controls also displayed a significant difference on day 10 (p=0.01).

Receiver operating curve analysis of the SAPS II score showed one of the highest discriminatory power between survivors and non-survivors with an area under the curve of 0.78. The sensitivity and specificity for the SAPS II score of 27 was 83% and 82%, respectively.

Survival:

Mortality trends analysis revealed a significant divergence between the C1-INH treated group and patients who received only standard treatment. The overall all-cause mortality rate in the control group was 45%, whereas the C1-INH group reached only 12% (p<0.008). The consistency of C1-INH effects on all-cause mortality (odds ratio 0.20; 95% confidence interval 0.06-0.71) was present even after adjustments for initial imbalances at admission.

The 28-day sepsis-related mortality in the C1-INH group was 8% compared to 45% in sepsis patients treated with only standard therapy (p=0.001). C1-INH administration showed the biggest absolute reduction risk of 70%, which was achieved in the subgroup of patients with SAPS II scores >27 (n=25). Absolute reduction risk in the subgroups of the lowest quartiles of C1-INH activity and patients with severe sepsis was among the highest. In sepsis patients with unchanged baseline, C1-INH activity (n=35), the mortality level was reduced from 55% in the control group to 17% in the treatment arm (p=0.04). In the subgroup of those who met severe sepsis criteria (n=39), mortality decreased from 57% in the control to 20% in the C1-INH group (p=0.03).

Kaplan-Meier survival curves displayed the mortality rate in the subgroups stratified by the parameters of sepsis severity. The most prominent benefit of C1-INH infusion was observed for sepsis-related mortality during 28 days of follow-up (p=0.001). The significant difference in survival seen in patients receiving C1-INH when compared to standard care without C1-INH was most relevant for patients with severe sepsis (p=0.03) and baseline SAPS II scores >27 (p=0.0001) and those who received vasopressors (p=0.01). A positive trend was observed in survival for patients with low C1-INH activity at study entry (p=0.06).

Discussion:

In the present study, we found that stratification of sepsis patients according to C1-INH activity could help identify those with a severe state due to a systemic inflammatory response. As we have previously reported in preliminary presentations in 2008-2010, quartile analysis of C1-INH activity at study commencement, before beginning treatment, revealed relative functional deficiency of C1-INH in some patients (Dolzhenkova et al., Crit. Care 12(Suppl 2):P382, 2008; Igonin et al., Crit. Care 13(Suppl 1):P363, 2009; and Igonin et al., Crit. Care 14(Suppl 1):P28). The data pointed to the possibility that sepsis patients who lack significantly elevated serum C1-INH activity but have increased complement activity and an increased level of CRP production are more likely to exhibit more severe disease. This finding agrees with the observation that an increase of C1-INH activity is part of a systemic response to infectious agents (Caliezi et al., Pharmacol. Rev. 52:91-112, 2000). In this vein, Hack et al. (Intensive Care Med. 19:S19-S28, 1993) suggested the concept of C1-INH “relative deficiency” in sepsis. In their study, the functional level of C1-INH activity was normal despite the fact that C1-INH is an acute-phase protein that, therefore, should have been elevated.

Patients with low or normal C1-INH may require additional C1-INH in generalized inflammatory states along with conventional treatment. Decades of experience with the replacement use of C1-INH for HAE display a remarkable safety record, but the doses used in HAE might not be enough to replace C1-INH functional deficiency found in some sepsis patients due to a high turnover of the protein during systemic inflammatory processes.

Previous authors have demonstrated various biological effects of C1-INH that may be relevant in severe infection. In vivo studies showed that direct interaction of C1-INH with lipopolysaccharide stimulation prevented endotoxin-induced shock and increased survival of animals modeling sepsis (Liu et al., Blood 105:2350-2355, 2005). C1-INH also reduced vascular permeability and lipopolysaccharide-mediated macrophage activation (Liu et al., Infect. Immun. 72:1946-1955, 2004). Increased bacterial uptake by phagocytes, reduction of viable Gram-negative bacteria found in blood, peritoneal fluid in cecal ligation sepsis models (Liu et al., J. Immunol. 179:3966-3972, 2007), and improved removal of bacteria via complement receptor 3-mediated pathway in pneumococcal meningitis in rats (Zwijnenburg et al., J. Infect. Dis. 196:115-123, 2007) were also proved. The experimental human endotoxemia study by Dorresteijn et al. (Crit. Care Med. 38:2139-2145, 2010) reported that C1-INH infusion reduced the level of proinflammatory cytokines, CRP and up-regulated release of the anti-inflammatory cytokine interleukin-10. Our data showed that down-regulation of the systemic inflammatory response in sepsis patients may be facilitated by C1-INH treatment.

A significant finding of the study was the association of C1-INH infusions at high doses with the improved survival of sepsis patients. Administration of C1-INH resulted in reduction in risk of death in the overall studied population with sepsis, and also in stratified patient subgroups based on severity parameters. The benefit of treatment with C1-INH expressed as adjusted odds ratio varied between subgroups was consistent with all-cause mortality over the 28-day study analyzed in the treatment arm and control.

Uncontrolled studies demonstrated survival benefits of C1-INH for critical care patients. Cumulative doses of 4000 U of C1-INH administered in 24 hours after emergency surgical revascularization to three patients resulted in weaning from aortic counterpulsation, decreased need for vasopressors, and may have contributed to the patients' full recovery (Bauernschmitt et al., Intensive Care Med. 24:635-638, 1998). In a case-control study where five patients with septic shock on mechanical ventilation and vasopressors received 3000 U of C1-INH during 12 hours, the authors reported a reduced requirement for vasopressors and absence of lethal outcomes upon follow-up (Hack et al., Lancet 339:378, 1992). Infusion of 6000 to 10,000 U of C1-INH during 12 hours in patients with streptococcal septic shock syndrome was associated with positive outcome in six out of seven cases (Fronhoffs et al., Intensive Care Med. 26:1566-1570, 2000).

One double-blind study did not show improved survival in the C1-NH-treated group of patients with severe sepsis, although they did find less severe organ dysfunction than in controls. Caliezi et al. (Crit. Care Med. 30:1722-1728, 2002) confirmed the renal-protective effect of C1-INH infused in high doses to sepsis patients along with standard care. The authors concluded that C1-INH dosage and the duration of treatment were not adequate for their cohort, failing to completely attenuate C4 b/c generation. In contrast to that trial, our study inclusion criteria limited the number of organ dysfunctions to not more than three. Not surprisingly, our subjects' baseline SAPS II score were less than those in Caliezi et at (supra) and C1-INH activity in our trial was significantly higher in sepsis patients at admission as well as during follow-up. Furthermore, the overall C1-INH activity increase registered longer in our treatment group by at least 1 day, than in the work of Caliezi et al. (supra).

Weaknesses that may have impacted the trial include the open-label design, which may contribute to observation bias. As well, slightly over half the patients enrolled were from two of the nine participating centers, which may reflect the positives and negatives of a potential homogeneous practice style. Also, the study was performed in two authorized but delayed subsequent sequences, and consecutive patients were included only within each stage. As mentioned, the severity of sepsis was tightly controlled with regard to upper limits. Lastly, other molecular data points were not gathered, such as active complement anaphylatoxins C3a and C5a, or other proinflammatory cytokines, such as interleukin-10. We attempted to minimize the open-label issue, in part, by choosing the objective categorical variable of 28-day mortality. To show wider applicability, a follow-up, randomized, double-blind, placebo-controlled trial should contain a larger number of consecutive patients, be multi-centered, from a wider geographic region, include wider representative causes of sepsis, and a wider range of sepsis severity, especially in higher severity. The trial should also gather a larger dataset of inflammatory markers. Further, a follow-up study with a larger number of subjects would help increase the power of the quartile analysis of severe sepsis vs. sepsis and mortality rate per quartile. Lastly, although it did not occur in this trial, block randomization may lead to significant imbalances that may confound trials. In our follow-up trial, we plan to stratify and/or permute randomization.

In conclusion, the clinical response profile and outcomes in sepsis patients treated with C1-INH could vary with respect to SAPS II score level as well as C1-INH activity. Diagnostically, sepsis patients with normal C1-INH activity and enhanced systemic inflammation at admission displayed worse severity of disease. A SAPS II score of >27 showed the best discriminatory power, and infusion of C1-INH had the highest impact on absolute mortality risk reduction. Regarding treatment, our study indicates that administration of human C1-INH may be considered a novel therapeutic approach in the treatment of sepsis. Future extended clinical trials may clarify if C1-INH is a predictive marker in sepsis progression and may definitively prove the favorable effect on survival by administration of C1-INH in patients suffering from sepsis.

### Example 2

### Effects of Purified Human C1-INH in Patients with Sepsis and ARDS

In the study below, we assessed the effects of high dose C1-INH infusion on pulmonary oxygenation and systemic inflammation in sepsis patients with ARDS. We studied 19 patients with sepsis and ARDS in surgical and medical intensive care units of nine university and city hospitals.

A retroactive analysis of a previous trial found a subpopulation of 19 patients who met both sepsis and ARDS criteria. All patients received conventional treatment. While seven patients were included in the control group, twelve patients had been randomized to also receive 12000 U of C1-INH infusion. Blood samples were drawn to measure C1-INH, C3, C4 and C-reactive protein (CRP) concentrations on days 1, 3, 5, 7, 10 and 28. Additionally, blood gases as well as the worst SpO2 were recorded on the same days.

Study Design:

In Example 1, we demonstrated that C1-INH infusion during the early phase of sepsis may be beneficial when assessed by 28-day survival. In that study, various population subsets presented with a different response to C1-INH treatment. Here, we describe and analyze the implication of C1-INH administration, and we assess the response to treatment for the subgroup of patients from the above trial diagnosed with sepsis and ARDS.

Of the 61 subjects participating in the earlier sepsis trial, a retrospective analysis identified a subgroup of 19 patients who entered the trial having developed sepsis that had already progressed to ARDS. These 19 patients are described in the current analysis. As above, we characterized sepsis, severe sepsis, and septic shock as defined by the American College of Chest Physicians/Society of Critical Care Medicine (1992). To confirm ARDS, we used the updated Berlin definition criteria: 1) acute onset within 1 week of a known clinical insult or new or worsening respiratory symptoms; 2) arterial blood gas ratio of PaO₂/FiO₂ (<100 for severe, <200 for moderate, <300 for mild ARDS (formerly referred to as ALI); 3) bilateral lung consolidation on chest X-ray; and 4) respiratory failure not explained by cardiac failure or fluid overload. Patients were included only if they were evaluated and began treatment within 48 hours of the sepsis onset. In this retroactive analysis of ARDS, 12 subjects were randomized into the C1-INH infusion arm, while the control arm contained seven patients. The treatment regime of C1-INH was chosen based on de Zwaan's study (European Heart Journal 23(21):1670-1677, 2002).

Treatment:

Patients from the treatment arm received 6,000 U in a single intravenous infusion of C1-INH immediately after randomization and 6000 U on the next day. The total dosage of C1-INH (Bicizar, BioGenius LLC, Moscow, Russia) was 12,000 Units during the initial 48 hours. All patients in the study were treated according to the standard of care protocols, regardless of C1-INH administration. This included, for example, a protective protocol of mechanical ventilation with low tidal volume and high positive end expiratory pressure to reduce FiO₂. The conservative fluid management was also applied excluding hemodynamically compromised patients. Fluid intake was adjusted to account for the saline used to dilute C1-INH in the total daily fluid replacement. All clinical, diagnostic and treatment decisions were at the discretion of attending physicians. The exclusion criteria was as above (see Example 1), and consent was obtained as described above.

Clinical and Laboratory Evaluation:

Vital signs were taken and haematological and biochemical assays were performed on days 1, 2, 3, 5, 7, 10, and 28 after randomization. Blood samples to determine serum C1-INH activity (Berichrom®-C1-inhibitor, Dade-Behring, Germany) and concentrations of complement subunits (C3 and C4) were also drawn on the above days. PaO₂ values were recorded on admission and the last day. The lowest SpO₂ values were recorded daily for all subjects. Patients whose oxygen saturation was 97% or lower were were later segregated. The first blood sample was obtained before the initiation of C1-INH treatment. SAPS score II was used as a surrogate marker of the illness severity. Mortality rate was assessed on day twenty-eight after randomization. Samples were analyzed at an independent laboratory.

Statistical Analysis:

Statistics were calculated and data were compared using the Mann-Whitney U and the paired Wilcoxon tests to assess any differences in outcome measures on the first and last day of the study. The last day in the study was defined as day 28 for those who survived the full follow-up period or the last day in the study of the non-survivors. Associations between variables were assessed by the Spearman's rank correlation coefficient. Qualitative comparisons were performed with Fisher's exact test. Twenty-eight day survival rates were calculated using Kaplan-Meier method. Statistical analyses were conducted with the GraphPad Prism statistical package (GraphPad Software).

Results:

Twelve patients with ARDS were randomized to the treatment arm and received C1-INH infusion, whereas seven patients with ARDS were enrolled as controls. Eighteen patients had hospital-acquired pneumonia and one had a soft tissue infection. The causative agent of hospital-acquired pneumonia was identified in 11 (58%) of all ARDS cases. In nine patients, Gram-negative strains were isolated either in blood or in sputum. One patient in the treatment group, suffering from acute lymphoblastic leukaemia, had Pneumocystis carinii as the cause of pneumonia while MRSA in combination with a Gram-negative strain was identified in the sputum of a second C1INH-treated subject. No organisms were identified in eight subjects. Carbapenems were the most often empirical antibiotic prescribed in both groups, 9 of 12 (75%) in the C1-INH group and 5 of 7 (71%) in control group (p=1.0).

Baseline clinical and laboratory parameters of the groups are shown in the Table below.

| | | | | | p (Fisher's |
|---|---|---|---|---|---|
| | C1-Esterase | | | | Exact |
| | Inhibitor Group | | Control Group | | Test) |
| Number (n) total | 12 | | 7 | | |
| (n = 19) | | | | | |
| Age, years | 56 | (24-70) | 53 | (24-64) | 0.5 |
| Gender, male/female | 5/7 | | 4/3 | | 0.4 |
| Hospital-acquired | 12 | (100%) | 6 | (86%) | 0.3 |
| pneumonia (HAP), n | | | | | |
| PaO2/FiO2, mm Hg | 193 | (121-270) | 256 | (120-284) | 0.1 |
| SpO2/FiO2 | 194 | (129-245) | 218 | (151-277) | 0.04 |
| C-reactive protein | 131 | (19-240) | 141 | (73-394) | 0.59 |
| (CRP), mg/l | | | | | |
| Mechanical | 9 | (75%) | 6 | (86%) | 1.0 |
| ventilation, n | | | | | |
| Simplified Acute | 34 | (16-51) | 33 | (25-42) | 0.9 |
| Physiology | | | | | |
| Score II (SAPS II) | | | | | |
| Septic shock, n | 5 | (42%) | 4 | (57%) | 0.6 |
| C1-esterase inhibitory | 1.6 | (1.2-3.0) | 1.5 | (0.8-2.28) | 0.7 |
| activity (C1INH, U/L) | | | | | |

Most of the clinical and laboratory characteristics between groups were similar and did not show any statistical difference on Day 1 before initiation of C1-INH infusion. All twelve patients treated with C1-INH presented with pneumonia (hospital-acquired) and sepsis. In the control group, all patients had sepsis, with six of seven having verified hospital-acquired pneumonia. Multiple trauma was the cause of hospital admission in three of the control patients. One patient with ARDS from the control group had a soft-tissue infection as the entry route. All patients were included in the study within 48 hours after onset of sepsis.

Patients from both groups had the same severity of state on Day 1, as assessed by SAPS II scores. Among the treated patients, 83% were on mechanical ventilation on admission whereas in the control group that number reached 86%. CRP as well as C1-INH activity were similar in both groups.

Infusion of human purified C1-INH resulted in the rapid increase of plasma C1-INH systemic activity in the treatment group compared to the values in the control group. On Day 2 of the study, the detected plasma C1-INH activity was significantly higher in the treatment arm (2.68 U/L; 1.86-3.68 U/L) than in the control arm (1.78 U/L; 1.01-2.27 U/L) (p=0.002). The same difference was observed on the third study day as well (p=0.01). In parallel, concentration of complement subunit C3 was restored in the C1-INH group. On the third study day after C1-INH infusion, C4 level was significantly higher, at 0.22 g/1 (0.14-0.34 g/l), than in the controls, at 0.11 g/l (0.07-0.26 g/l; p=0.02). By day 5 the difference between the groups was not significant for C1NH activity (p=0.1) or for C4 (p=0.3). A trend could be observed of lowered values of CRP after C1-INH infusion. The CRP concentration in the treatment group on Day 5 reached 117 mg/l (9.2-190.6 mg/l) whereas the control arm concentration was 180 mg/1 (127-255 mg/l) (p=0.06).

Overall, PaO₂/FiO₂ significantly increased in the C1-INH treated arm, from the initial day's median of 193 mm Hg (121-270 mm Hg) to the day 5 observed median of 235 mm Hg (107-525 mm Hg; p=0.03) and to the last day of observation median of 286 mm Hg (164-980 mm Hg; p=0.03). A similar significant elevation of SpO₂/FiO₂ ratio (p=0.007) accompanied the infusion of C1-INH. That trend was not observed in the PaO2/FiO2 (p=0.15) and SpO2/FiO2 (p=0.1) dynamics in the control. This was interesting, considering that the PaO₂/FiO₂ ratio in the control arm of 256 (120-284) was higher on admission in comparison to 193 (121-270) in the treatment arm and similarly the SpO₂/FiO₂ ratio in the control arm of 245 (151-277) was higher on admission in comparison to 194 (129-245) in the treatment arm (p=0.04). The correlation analysis revealed a significant positive correlation between plasma C1-INH activity and SpO₂/FiO₂ on admission (r=0.37; p=0.005).

To account for the prior validated correlation values between PaO₂/FiO₂ and SpO₂/FiO₂ and the flattening curve of the upper portion of the oxyhemoglobin dissociation curve, PaO₂/FiO₂ and SpO₂/FiO₂ ratios were also calculated for the 16 subjects whose oxygen saturation were 97% or lower.

The number of patients with non-ARDS/mild/moderate ARDS on day 1 before C1INH infusion in the treatment arm was 0/517 (n=12) and in the control—0/6/1 (n=7) whereas on the last day of the study in the treatment arm 4/5/3 and control group—1/2/4, respectively. Four patients in the treatment group had no signs of ARDS on the last day of their participation in the study, none patients recovered from ARDS in control. Moreover, the difference of those who worsen the ARDS grade on their last day in the study reached the level of significance. In the treatment group, not one of 12 patients worsened in ARDS severity grade, whereas in the control group 3 of 4 patients changed from mild to moderate severity (p=0.036).

Overall, all-cause mortality over the 28 days of the study was 33% in the treatment group versus 71% in the control arm with an Absolute Reduction Risk (ARR) of 38%. The difference in the improved 28-day survival trend in the C1-INH infused arm was reflected by Kaplan-Meier curve (ρ=0.1 Log Rank Mantel-Cox).

In summary, the subpopulation of sepsis patients with ARDS treated with high-doses of C1-INH showed significant improvement in blood oxygenation, as well as a positive survival trend. Significantly, in the treatment group, C1-INH activity rose to 2.68 U/L (1.86-3.68 U/L) compared to 1.78 U/L (1.01-2.27 U/L; p=0.002) in the control arm; C4 levels rose to 0.22 g/l (0.14-0.34 g/l) compared to 0.11 g/l (0.07-0.26 g/l; p=0.03) in controls; and the CRP level dropped to 117 mg/l (9.2-190.6 mg/l) compared to 180 mg/1 (127.5-254.7 mg/l; p=0.05) in the control group. Infusion of C1-INH resulted in improved blood oxygenation, with the initial PaO₂/FiO₂ ratio of 193 mm Hg (121-270 mm Hg) ascending on the last day of observation to a median of 286 mm Hg (164-980 mm Hg; p=0.03). For the 16 patients with SpO₂/FiO₂ on admission of <97% a similarly significant (p=0.04) association was detected. At the same time, C1-INH treatment was associated with the 28-day all-cause mortality absolute reduction risk of 38%.

Discussion:

The most significant finding of this retrospective analysis suggests that administration of human purified C1-INH to patients with ARDS and sepsis may improve gas exchange and decrease the degree of hypoxemia. This association was shown by the positive correlation of plasma C1-INH activity and oxygenation ratios. A substantial increase of PaO₂/FiO₂ was observed in the group of patients who received C1-INH infusion whereas those on the standard treatment protocol had a trend to lower median values on the last day of follow-up. Similarly, the control arm started with a higher SpO₂/FiO₂ than did the treatment arm, and the treatment arm patients with “relative” C1-INH insufficiency were should suffer more from severe hypoxia as indicated by correlation coefficient and from sustained systemic inflammatory response reflected by CRP plasma level. However, replacement with C1-INH infusion may have down-regulated mediator response and stabilized oxygen supply, as evidenced by a significantly higher PaO₂/FiO₂.

Our results suggest that C1-INH with its multiple functionality may act as a pulmonary protector in ARDS by containing both local and systemic inflammatory response and by controlling vascular permeability. C1-INH may act as a multiple serine protease inhibitor. The normal range of C1-INH plasma activity is calculated according to serine protease inhibition. We postulate that the major anti-inflammatory effects of C1-INH administered at high doses (>100 U/kg) may be linked to its unique N-terminal domain, which is not responsible for protease inhibition. If this were the case, high doses of C1-INH may be beneficial in ARDS patients with preserved C1-INH levels. Moreover, if that were so, then one might think that administering purified C1-INH in ARDS subjects with depleted endogenous C1-INH would produce more considerable positive effects utilizing altered inhibitory pathways.

Our results indicate possible lung protective effects of high-dose delivery of C1-INH. Kluge et al. gave a total dose of 12,000 U of C1-INH administered intravenously over 48 hours in a case of severe ARDS due to pneumonia, which led to a decrease of the extravascular lung water index from 30 ml/kg to 15 ml/kg and an increase of PaO₂/FiO₂ from 59 mm Hg to 119 mmHg over several days (Kluge et al., Intensive Care Med 30(4):731, 2004). In two cases of graft failure after lungs transplantation, which presents pathogenically very similar to ARDS, C1-INH was infused to treat severe capillary leak syndrome associated with massive pleural effusion. The C1-INH administration was associated with a decrease in pleural effusion and improvement in pulmonary gas exchange (Strüber, Intensive Care Med 25(11):1315-1318, 1999).

Although the specific mechanism by which C1-INH affects ARDS is not yet well defined, improved gas exchange in ARDS might be related to decreased fluid leakage. Down-regulation of activated neutrophils and elastase release by C1-INH infusion in patients with severe sepsis were determined in the double-blinded randomized placebo-controlled study of Zerleeder et al. (Clin. Diagnos. Lab Immunol. 10(4):529-535, 2003). Patients with hereditary low C1-INH activity demonstrated up-regulated thrombin production (Cugno et al., Blood 89(9):3213-3218, 1997). Purified C1-INH might inhibit increased thrombin activity on endothelium via selectins (Caccia et al., Blood Coagul. Fibrinolysis 22(7):571-575, 2011). This systemic inflammatory response was also down-regulated by purified C1-INH, which was reflected by reduced levels of proinflammatory cytokines, CRP and increased anti-inflammatory cytokine interluekin-10 in the experimental human endotoxemia study by Dorresteijn et al. (Crit. Care Med. 38(11):2139-2145, 2010).

The recently found connection between coagulation cascade and complement system might represent an additional insight to the suggested protective effect of C1-INH administration on respiratory system in ALI. As an example, in the ALI animal model, thrombin activated cleavage of C5 to C5a in the absence of C3 (Huber-Lang et al., Nature Med. 12(60):682-687, 2006). Furthermore, thrombin was also capable of cleaving C3 to C3a in vitro. Thrombin-antithrombin complex showed positive significant correlation with C5a in the early course of trauma patients (Amara et al., J. Immunol. 185(9):5628-5636, 2010). In another work, mannose-binding protein associated serine protease 2 (MASP2) triggered generation of thrombin from the precursor prothrombin (Krarup et al., PLoS ONE 2(7):e623, 2007). Such cross-talk of the cascades could facilitate the increased production of anaphilotoxins C3a and C5a attributed to immunoparalysis and multi-organ dysfunction in sepsis (Huber-Lang et al., J. Immunol. 166(2):1193-1199, 2001). The collective attributes of C1-INH to produce the full blockade of the complement system, directly interact with thrombin, as well as indirectly inhibit the intrinsic pathway of coagulation, may result in the subsequent effective interference of these cascades. The above-mentioned interplay of C1-INH explains the clinical data and reported multiple pathogenetically relevant effects of that protein in sepsis and ARDS.

The results of our randomized study showed a significant association of the improved survival of sepsis patients with high doses of C1-INH. However, because of the heterogeneity of the sepsis population and clinical responses, the advantages of C1-INH use are likely to vary in sepsis subgroups. In our analysis, administration of exogenous C1-INH along with restoration of C1-INH activity resulted in ARR of 38% of patients with ARDS and sepsis. All-cause 28-day mortality displayed a lower trend for those ARDS patients with sepsis who were treated with C1-INH.

One weakness of this particular analysis is that the initial trial was designed to examine sepsis in general, not ARDS in particular. As such, some of the instruments (and frequency of measurements) commonly found in adult ARDS trials were not utilized. One important example of this is that two additional subjects who survived ARDS but were excluded from our analysis. They fit all the criteria of the Berlin definition on ARDS, yet their initial PaO₂ could not be confirmed, and they came to us on mechanical ventilation. We had numerous readings of their SpO₂/FiO₂, which were low (one subject with a reading of 196, and one subject with a reading of 307). We long considered adding these two subjects into our analysis, using the cut-offs of SpO₂/FiO₂ of <235 for ARDS, and <315 for ALI mentioned as potential substitute proxy values (Rice et al., Chest 132(2):410-417, 2007). Since pulse oximetry is a more frequently used measure, it seemed plausible to include these subjects, especially since their overall clinical picture showed respiratory collapse, which may have been confirmed by initial PaO₂. Using those two subjects in our analysis, both of whom survived, would lead to several more statistically significant findings, even in this small group analysis. Sadly, the issue of the use of PaO₂ versus SpO₂ is more dire when considering children in whom taking a non-invasive blood gas reading may make more sense and be an acceptable proxy, or in developing countries, where resources are limited (Thompson, Crit. Care Med. 35(2 Suppl):52-10, 2007).

From the 19 subjects that we analyzed, and to add to the body of knowledge, we further report here SpO₂ alongside PaO₂ for the 16 subjects whose oxygenation saturation was 97% or lower because at the upper portion of the oxyhemoglobin dissociation curve, the curve flattens out, and may be misleading. Assessing data from only these 16 subjects, the SpO₂ mirrors carefully the PaO₂ findings, and still shows statistical significance.

The fact that we are assessing a small sample size means that we have most likely an underpowered analysis. For example, the weakness of the mortality analysis may have been related to the small number of observed cases. Lastly, an underpowered analysis may contribute to observation bias.

C1-INH exhibits a wide spectrum of biological effects. For example, it modulates the Kallikrein-Kinin system and prevents capillary leakage, impacts leukocyte activation (Wuillemin et al., Blood 85(6):1517-1526, 1995; Eriksson and Sjogren, Immunology 86(2):304-310, 1995), and enhances bactericidal activity. It was recently suggested that increased C1-INH activity may be protective, whereas low levels of C1INH may be considered a risk factor for contact system-mediated undesirable effects (Zhou et al., PLoS ONE 7(4):e34978, 2012). Long-term clinical observation demonstrated the successful prevention and inhibition of angioedema in terms of C1-INH hereditary deficiency (De Serres et al., Transfusion and Apheresis Science 29(3):247-254, 2003). We hypothesized that since C1-INH reduces capillary leakage and demonstrates sustained anti-inflammatory properties, it might have a protective effect on the lungs and improve gas exchange in cases of ARDS. In fact, in a single case report, the decrease of extravascular lung water index and reduction of FiO₂ was associated with C1-INH administration (Kluge and Kreymann, Intensive Care Med. 30(4):731, 2004).

In conclusion, this is the first observation in which the pharmacodynamics benefits of C1-INH treatment were determined along with the positive survival trend of patients with ARDS due to hospital-acquired pneumonia complicated with sepsis. C1-INH represents a unique endogenous modulator of key cascades and multiple molecules implicated in the mechanisms of local and systemic organ injuries in sepsis and ARDS. The protective lung effects of high dose therapy with C1-INH should be investigated in future multi-institutional randomized clinical trials to determine which subpopulations might benefit from its administration.

What is claimed is:

1. A method of assessing the protective capacity of endogenous C1-INH in a patient who has been diagnosed with acute respiratory distress syndrome, the method comprising:
(a) providing a fluid sample from the patient;
(b) determining the amount of C1-esterase inhibitor (C1-INH) functional activity in the sample; and
(c) comparing the amount of C1-INH functional activity to a reference standard, wherein a level of C1-INH functional activity comparable to that within a healthy patient population indicates that the patient's own protective capacity is compromised.

2. The method of claim 1, wherein the amount of C1-INH functional activity is less than or about 1.70 U/L.

3. The method of claim 1, wherein the amount of C1-INH functional activity is less than or about 1.34 U/L.

4. The method of claim 1, wherein the amount of C1-INH functional activity is about 1.34 to 1.70 U/L.

5. The method of any of claims 1-4, further comprising the step of administering to the patient a therapeutically effective amount of C1-INH, wherein the amount is about 12,000 IU administered as two, 6,000 IU doses within about a 24-48 hour period.

6. A method of treating ARDS in a patient with C1-INH functional activity below about 1.7 U/L, the method comprising administering to the patient a total dose of C1-INH of 3,000 IU, 6,000 IU, 9000 IU, 12,000 IU, or 16,000 IU in single or divided doses.

7. A method of treating ARDS in a patient with C1-INH functional activity below about 1.7 U/L, the method comprising administering to the patient about 1,000, 2,000, or 3,000 IU C1-INH about every 12 hours.

8. A method of treating ARDS in a patient with C1-INH functional activity below about 1.7 U/L, the method comprising administering to the patient about 50-250 U/kg C1-INH as a single dose or about 1.25-3 U/kg/hour C1-INH as an infusion.

9. The method of any of claims 5-8, further comprising the step of identifying a patient in need of treatment.

10. The method of claim 9, wherein the patient is a human patient.

11. The method of any of claims 5-9, wherein the C1-INH is administered intravenously.

12. The method of any of claims 5-9, wherein the C1-INH is administered by insufflation, subcutaneously, intracutaneously, intranasally, intratracheally, topically, intracavitally, or intraspinally.

13. The method of any of claims 5-9, wherein the C1-INH is delivered in combination with a second pharmaceutical agent.

14. The method of claim 13, wherein the second pharmaceutical agent is Protein C, activated protein C, antithrombin III, rituximab, eculizumab, or IVIG.

15. The method of claim 13, wherein the second pharmaceutical agent is complement receptor agonist or antagonist.

16. The method of claim 15, wherein the second pharmaceutical agent is a kallikrein inhibitor or a bradykinin receptor inhibitor.

17. The method of claim 13, wherein the second pharmaceutical agent is an antibiotic, steroid, or fresh frozen plasma.

18. A purified fragment of C1-INH, wherein the fragment comprises the serpin domain or the N-terminal domain.

19. The purified fragment of C1-INH of claim 18, wherein the N-terminal domain comprises the N-terminal 116 amino acid residues.

20. A method of treating ARDS in a patient with C1-INH functional activity above 1.7 U/L, the method comprising administering to the patient C1-INH with an inactivated serpin domain.

21. A kit comprising instructions for use in methods of assessing the protective capacity of endogenous C1-INH in a patient and one more of the following items:
(a) a fluid sample comprising a reference standard;
(b) C1-INH;
(c) a second therapeutic agent; and
(d) paraphernalia for delivery of the C1-INH and/or the second therapeutic agent to the patient.

22. A method of assessing the protective capacity of endogenous C1-INH in a patient who has been diagnosed with sepsis or a sepsis-related condition, the method comprising:
(a) providing a fluid sample from the patient;
(b) determining the amount of C1-esterase inhibitor (C1-INH) functional activity in the sample; and
(c) comparing the amount of C1-INH functional activity to a reference standard, wherein a level of C1-INH functional activity comparable to that within a healthy patient population indicates that the patient's own protective capacity is compromised.

23. The method of claim 22, wherein the amount of C1-INH functional activity is less than or about 1.70 U/L; less than or about 1.34 U/L; or about 1.34-1.70 U/L.

24. The method of claim 22 or 23, further comprising the step of administering to the patient a therapeutically effective amount of C1-INH, wherein the amount is: (a) about 12,000 IU administered as two, 6,000 IU doses within about a 24-48 hour period; (b) about 3,000 IU, 6,000 IU, 9000 IU, 12,000 IU, or 16,000 IU in single or divided doses; (c) about 1,000, 2,000, or 3,000 IU C1-INH about every 12 hours; or (d) about 50-250 U/kg C1-INH as a single dose or about 1.25-3 U/kg/hour C1-INH as an infusion.

25. The method of claim 22, further comprising the step of identifying a patient in need of treatment.

26. The method of claim 25, wherein the patient is a human patient.

27. The method of claim 24, wherein the C1-INH is administered intravenously, by insufflation, subcutaneously, intracutaneously, intranasally, intratracheally, topically, intracavitally, or intraspinally.

28. The method of claim 24, wherein the C1-INH is delivered in combination with a second pharmaceutical agent.

29. The method of claim 28, wherein the second pharmaceutical agent is Protein C, activated protein C, antithrombin III, rituximab, eculizumab, or IVIG; a complement receptor agonist or antagonist; a kallikrein inhibitor or a bradykinin receptor inhibitor; or an antibiotic, steroid, or fresh frozen plasma.

30. A method of treating sepsis or a sepsis-related condition in a patient with C1-INH functional activity above 1.7 U/L, the method comprising administering to the patient C1-INH with an inactivated serpin domain.

31. A method of assessing the protective capacity of endogenous C1-INH in a patient who has been diagnosed with a burn, a burn-related condition, or another traumatic injury, the method comprising:
(a) providing a fluid sample from the patient;
(b) determining the amount of C1-esterase inhibitor (C1-INH) functional activity in the sample; and
(c) comparing the amount of C1-INH functional activity to a reference standard, wherein a level of C1-INH functional activity comparable to that within a healthy patient population indicates that the patient's own protective capacity is compromised.

32. The method of claim 31, wherein the amount of C1-INH functional activity is less than or about 1.70 U/L; less than or about 1.34 U/L; or about 1.34-1.70 U/L.

33. The method of claim 31 or 32, further comprising the step of administering to the patient a therapeutically effective amount of C1-INH, wherein the amount is: (a) about 12,000 IU administered as two, 6,000 IU doses within about a 24-48 hour period; (b) about 3,000 IU, 6,000 IU, 9000 IU, 12,000 IU, or 16,000 IU in single or divided doses; (c) about 1,000, 2,000, or 3,000 IU C1-INH about every 12 hours; or (d) about 50-250 U/kg C1-INH as a single dose or about 1.25-3 U/kg/hour C1-INH as an infusion.

34. The method of claim 33, further comprising the step of identifying a patient in need of treatment.

35. The method of claim 34, wherein the patient is a human patient.

36. The method of claim 33, wherein the C1-INH is administered intravenously, by insufflation, subcutaneously, intracutaneously, intranasally, intratracheally, topically, intracavitally, or intraspinally.

37. The method of claim 33, wherein the C1-INH is delivered in combination with a second pharmaceutical agent.

38. The method of claim 46, wherein the second pharmaceutical agent is Protein C, activated protein C, antithrombin III, rituximab, eculizumab, or IVIG; a complement receptor agonist or antagonist; a kallikrein inhibitor or a bradykinin receptor inhibitor; or an antibiotic, steroid, or fresh frozen plasma.

39. A method of treating a burn, a burn-related condition, or another traumatic injury in a patient with C1-INH functional activity above 1.7 U/L, the method comprising administering to the patient C1-INH with an inactivated serpin domain.

40. A method of assessing the protective capacity of endogenous C1-INH in a patient who has been diagnosed with Stevens-Johnson Syndrome (SJS) or who is suffering from complications following a coronary artery bypass graft surgery, the method comprising:
(a) providing a fluid sample from the patient;
(b) determining the amount of C1-esterase inhibitor (C1-INH) functional activity in the sample; and
(c) comparing the amount of C1-INH functional activity to a reference standard, wherein a level of C1-INH functional activity comparable to that within a healthy patient population indicates that the patient's own protective capacity is compromised.

41. The method of claim 40, wherein the amount of C1-INH functional activity is less than or about 1.70 U/L; less than or about 1.34 U/L; or about 1.34-1.70 U/L.

42. The method of claim 40 or 41, further comprising the step of administering to the patient a therapeutically effective amount of C1-INH, wherein the amount is: (a) about 12,000 IU administered as two, 6,000 IU doses within about a 24-48 hour period; (b) about 3,000 IU, 6,000 IU, 9000 IU, 12,000 IU, or 16,000 IU in single or divided doses; (c) about 1,000, 2,000, or 3,000 IU C1-INH about every 12 hours; or (d) about 50-250 U/kg C1-INH as a single dose or about 1.25-3 U/kg/hour C1-INH as an infusion.

43. The method of claim 40, further comprising the step of identifying a patient in need of treatment.

44. The method of claim 43, wherein the patient is a human patient.

45. The method of claim 42, wherein the C1-INH is administered intravenously, by insufflation, subcutaneously, intracutaneously, intranasally, intratracheally, topically, intracavitally, or intraspinally.

46. The method of claim 42, wherein the C1-INH is delivered in combination with a second pharmaceutical agent.

47. The method of claim 46, wherein the second pharmaceutical agent is Protein C, activated protein C, antithrombin III, rituximab, eculizumab, or IVIG; a complement receptor agonist or antagonist; a kallikrein inhibitor or a bradykinin receptor inhibitor; or an antibiotic, steroid, or fresh frozen plasma.

48. A method of treating Stevens-Johnson Syndrome (SJS) or complications following a coronary artery bypass graft surgery, in a patient with C1-INH functional activity above 1.7 U/L, the method comprising administering to the patient C1-INH with an inactivated serpin domain.

49. A method of assessing a patient who has been diagnosed with acute respiratory distress syndrome (ARDS), the method comprising:
(a) assessing the protective capacity of endogenous C1-INH in the patient, wherein the assessment of C1-INH comprises (i) providing a fluid sample from the patient and (ii) determining the amount of C1-esterase inhibitor (C1-INH) functional activity in the sample;
(b) assessing the severity of hypoxemia in the patient, wherein the assessment of hypoxemia comprises measuring the ratio of partial pressure arterial oxygen to the fraction of inspired oxygen (PaO2/FiO2 level); and
(c) comparing the amount of C1 INH functional activity to a reference standard, wherein a level of C1 INH functional activity comparable to that within a healthy patient population and a PaO2/FiO2 level below 300 mm Hg indicate that the patient's own protective capacity and gas oxygenation are compromised.

50. The method of claim 49, wherein the amount of C1 INH functional activity is less than or about 1.70 U/L and the PaO2/FiO2 level is below 300 mm Hg.

51. The method of claim 49, wherein the amount of C1 INH functional activity is less than or about 1.34 U/L and the PaO2/FiO2 level is below 300 mm Hg.

52. The method of claim 49, wherein the amount of C1 INH functional activity is about 1.34 to 1.70 U/L and the PaO2/FiO2 level is below 300 mm Hg.
